(19) United States
(12) Patent Application Publication
(10) Pub. No.: US 2018/0035961 A1
(43) Pub. Date: Feb. 8, 2018

(54) MOBILE GRATING-DETECTOR ARRANGEMENT

(71) Applicant: SIEMENS HEALTHCARE GMBH, ERLANGEN (DE)

(72) Inventors: PETER BARTL, ERLANGEN (DE); MARCUS RADICKE, VEITSBRONN (DE); LUDWIG RITSCHL, ERLANGEN (DE); SVEN-MARTIN SUTTER, HERZOGENAURACH (DE); THOMAS WEBER, HAUSEN (DE); MARTINO LEGHISSA, WIESENTHAU (DE); JOSEF ZEIDLER, MARKTREDWITZ (DE)

(21) Appl. No.: 15/671,586

(22) Filed: Aug. 8, 2017

(30) Foreign Application Priority Data
Aug. 8, 2016 (DE) 10 2016 214 678.5

(51) Int. Cl. A61B 6/00; G01J 3/18; G01N 23/20; A61B 6/08; G03B 42/02

(52) U.S. Cl. CPC A61B 6/484; A61B 6/08; A61B 6/4291; G03B 42/02; G01N 23/20; G01J 3/1833; G21K 2207/005; G01J 2003/1842

(57) ABSTRACT

A mobile grating-detector arrangement has an X-ray detector and at least one grating. The grating-detector arrangement is configured to record an interferometric X-ray image of at least one body part of a patient in a patient bed in operation. In addition, an X-ray system with such a grating-detector arrangement and its use for X-ray interferometric imaging is described.

### CROSS-REFERENCE TO RELATED APPLICATION

This application claims the priority, under 35 U.S.C. §119, of German application DE 10 2016 214 678.5, filed Aug. 8, 2016; the prior application is herewith incorporated by reference in its entirety.

### BACKGROUND OF THE INVENTION

### Field of the Invention

The invention relates to a mobile grating-detector arrangement.

The relatively young technology of interferometric X-ray imaging is a variant of phase contrast imaging. It is usually based on one, two or three gratings G0, G1 or G2 being introduced into the beam path of a customary X-ray arrangement in an X-ray imaging system. This is shown in FIG. 7. For small X-ray foci (e. g. microfocus X-ray tubes), the absorption grating G0 is unnecessary as the X-ray radiation is already coherent in location. It can be omitted in this case. However, if the X-ray focus is over a certain size, the absorption grating G0 is necessary and as a rule arranged close to the beam path of an X-ray source 41. The absorption grating G0 is customarily structured such that the coherence of the X-rays is generated by a parallel, equidistant structuring of the absorption grating G0 in one direction. However, the absorption grating G0 can also be chessboard-like in structure so that the necessary coherence is provided in two orthogonal directions. The spacing of the grating structure is regularly selected such that the Lau effect is met in the image plane. In other words, the spacing between the absorption grating G0 and a detector 2 arranged in the image plane is selected in relation to the spacing inside the grating structure such that a constructive superimposition of the diffracted X-rays is produced on the absorption grating G0 in the image plane.

A grating G1 is customarily designed as phase grating G1. With the phase grating G1, advantage is taken of the so-called Talbot effect. This generates a self-image at certain intervals to the phase grating G1 of the phase grating G1. If an object for examination, for example, a patient P, is now introduced into the beam path, it interferes with the self-image of the phase grating G1. Various image information can be obtained from these interferences hereinafter, namely absorption, differential phase shift and dark field.

However, the resolution of a customary medical X-ray detector 2 is generally insufficient to read out the interference pattern. It is therefore necessary for a further grating to be introduced into the beam path as an analyzer grating G2. The grating period of this grating is configured to conform to the undisturbed self-image of the phase grating G1. In the context of so-called “phase stepping”, customarily the analyzer grating G2 is gradually shifted towards the phase grating G1, an X-ray image being recorded after each step. The step size is selected such that at least three images are recorded between a period of the undisturbed interference image and the phase grating G1. In addition, an image of the grating without the patient P in the beam path and an image with the patient P in the beam path is customarily recorded. The aforementioned image information can now be obtained from the images thus recorded.

With the X-ray interferometry examinations known hitherto, the patient must, for example, be placed in a standing position in a special X-ray room or on a special X-ray couch. However, there is also a requirement, for example, in an intensive care unit, to examine patients who are unable to leave their beds. In the case of such patients especially, current research findings indicate that thorax images recorded by means of X-ray interferometric imaging have additional clinical value for diagnosis. However, with interferometric X-ray imaging, in contrast to standard methods of customary X-ray imaging by means of absorption, it is not sufficient only to position a relatively flat detector under the patient in the bed on account of the necessary grating.

### SUMMARY OF THE INVENTION

It is therefore the object of the present invention to specify a device which makes interferometric X-ray imaging easier in the case of immobile patients.

The aforementioned grating-detector arrangement is mobile and has an X-ray detector and at least one grating. It is configured to record an X-ray interferometric image of a patient in a patient bed in operation.

Here “mobile” means that the grating-detector arrangement can be transported without additional aids. In other words, it can, for example, be driven or carried. Compared to known systems for interferometric X-ray imaging, it is not permanently installed by way of any connecting elements, hence for example, a base or a track system, i.e. permanently connected to the components of an examination area. The grating-detector arrangement can preferably also be positioned in relation to the patient bed and the patient therein such that it can be arranged as part of an X-ray system with a defined position, hence at a defined distance and with a defined orientation, with regard to an X-ray source.

The at least one grating is a grating which in terms of structure and material is suitable to influence incident X-rays such that the reflected X-rays have a spatially arranged, preferably spatially periodic pattern of intensity distribution. The pattern of intensity distribution is generally dependent on the distance from the grating. A grating is preferably configured as a phase grating. It is preferably arranged as close as possible to the area or body part of the patient for examination. For as the distance of the phase grating from the region of interest of the patient increases, the quality of the image information obtained from the patient decreases.

Depending on the embodiment of the X-ray system for which the grating-detector arrangement is configured, the grating-detector arrangement contains further grating. Thus, for example, an absorption grating, as aforementioned, is arranged in the beam path close to the X-ray source. The absorption grating generates the necessary coherence of the X-rays if the X-ray source is not already emitting coherent rays on its own. Furthermore, an analyzer grating is preferably arranged at a defined distance from the phase grating and in the vicinity of the detector between the detector and the phase grating to read out the image information. With the aid of the analyzer grating, the image information to be obtained can be read out in the context of the aforementioned phase stepping, even if the detector would be otherwise unsuitable for this due to its resolution.

Preferably, the grating and the detector are essentially parallel and arranged such that the centers of their surfaces lie on a common line corresponding to a central beam of a beam path of the emitted radiation of the X-ray source in the case of an X-ray system.

The patient for examination may, for example, also be an animal patient. Preferably, however, a person is examined as a patient. Accordingly, the patient bed is preferably a customary hospital bed or a bed like those used for intensive care.

Unlike previous X-ray interferometers, which are unsuitable for mobile examinations of patients in their beds, a patient can thus be examined in their bed using the grating-detector arrangement according to the invention and need not be moved to a corresponding examination table or be forced into a standing position.

Besides a grating-detector arrangement according to the invention, the aforementioned X-ray system also has an X-ray source. The X-ray source is preferably arranged such that a central beam of the X-ray radiation emitted hits the surface of the grating or the surface of the X-ray detector at a right angle. Hence, the X-ray source generates the radiation which, in the context of the examination, penetrates the patient as an object for examination, is scattered on the structures and materials of their body parts or organs and is finally detected as an X-ray image of the patient with the aid of the detector. However, the X-ray image is not a customary X-ray image in the sense of a simple absorption image but an interference pattern of the phase grating disrupted by the patient, with the aid of which the scattering structures, i.e. the structures causing the interference, can be deduced in comparison with the undisturbed interference pattern. With the aid of the X-ray system according to the invention, interferometric X-ray imaging with a grating-detector arrangement according to the invention is hence enabled.

As aforementioned, a grating-detector arrangement according to the invention is used for X-ray interferometric imaging, preferably for thorax imaging, for a patient in the patient bed. Interferometric imaging of the thorax region of the patient is particularly advantageous as it offers added clinical value for the diagnosis, in particular for the organs located there.

Further, particularly advantageous embodiments and developments of the invention result from the dependent claims and the following description, wherein the independent claims of a claim category can also be developed analogously to the dependent claims of another claim category and in particular, individual features of different exemplary embodiments or variants of new exemplary embodiments or variants can also be combined.

The grating-detector arrangement according to the invention must preferably be positioned between a flat, folded position and an unfolded position. At least some of the grating is arranged in the unfolded position at defined intervals, hence preferably at least two gratings at a defined interval.

In the flat, folded position, the grating-detector arrangement can advantageously be pushed between the patient and patient bed with ease. Due to the flat embodiment of the grating-detector arrangement in the folded position, the patient need only be raised a little or the mattress of the patient bed can be pushed in a little so that the grating-detector arrangement can be inserted into the resulting intermediate space. Between the folded position and the unfolded position, the components of the grating-detector arrangement can, for example, be repositioned by means of a mechanical lifting or adjusting device but also, for example, hydraulically, pneumatically or by electric motors. This can, for example, take place in a linear movement such that the defined interval is produced between the gratings in the unfolded position.

Preferably, however, this is a swivel mechanism with which a supporting plate as a supporting surface for the patient with respect to a base frame is swiveled around a pivot bearing, for example, around a hinge. As part of the swiveling procedure, hence during positioning between the folded position and the unfolded position, the gratings are also moved simultaneously such that, as described, they are also at a defined interval in the unfolded position. This swivel mechanism is particularly advantageous as the swivel movement mimics the assembly of the head end of the patient bed. In other words, for a patient in the bed this movement also represents a familiar sequence of movements. It is even customary for patients to be supported in a slightly seated position in an intensive care unit, such that the introduction of the grating-detector arrangement is also possible within the limits set by any intensive care connections for the care of the patient.

A grating-detector arrangement according to the invention preferably contains a first grating and a second grating. The first grating, the second grating and the X-ray detector are arranged in parallel, at least in the unfolded position. For at least in the unfolded position, a parallel arrangement of the grating and the detector for the X-ray interferometric imaging is necessary. Particularly preferably the grating and the X-ray detector are also located in intermediate positions between the folded position and the unfolded position in a parallel arrangement. If the support of the patient is only possible within a very limited context, in this way X-ray interferometric imaging can also already take place advantageously in the intermediate positions.

A grating-detector arrangement according to the invention is preferably connected to a trolley and with its aid can be positioned in relation to the patient. The trolley preferably contains casters and, if applicable, other means of positioning such as, for example, swivel joints, linear adjustments or folding mechanisms. It can therefore be moved with the aid of the casters and thus be transported to the respective patient bed. By the patient bed the orientation of the grating-detector arrangement in relation to the patient or to the patient bed then takes place by way of the additional positioning means.

A grating-detector arrangement according to the invention is preferably connected to a supporting plate for the patient by way of the trolley. The supporting plate preferably consists of a material which is transparent for X-rays, for example, Plexiglas or the like. With the supporting plate, the patient can thus be suitably positioned without the supporting plate having an influence on X-ray imaging. With corresponding support of the patient, the grating-detector arrangement can now be positioned in front of or behind the patient as preferred or according to circumstances. The grating-detector arrangement according to the invention thus advantageously creates increased flexibility in its positioning in relation to the patient.

Besides a grating-detector arrangement and an X-ray source, an X-ray system according to the invention preferably also has a separate supporting plate for patients which is connected to the patient bed in operation. Unlike the aforementioned exemplary embodiments, the supporting plate is therefore not part of the grating-detector arrangement or also not connected to the grating-detector arrangement by way of a trolley. It is individually attached to the patient bed as part of the X-ray system in order to position the patient for recording.

The supporting plate particularly preferably has—as later described—markers with which it can be positioned in relation to the rest of the X-ray system. By means of the separate supporting plate, the remaining components of the X-ray system can be advantageously positioned completely independently of the positioning of the patient. The supporting plate has suitable means of attachment for attachment to the patient bed such as, for example, clamps or similar positive-fit and/or friction-type elements. The supporting plate preferably has a suitable adjustment mechanism for positioning of the patient such as, for example, a scissor-type lift, a hydraulic or pneumatic lift, or an electrically operated variable displacement motor.

In a grating-detector arrangement according to the invention, the supporting plate preferably has markers to position the patient and the grating-detector arrangement in relation to an X-ray source. The markers are particularly preferably detected by a detection unit which is arranged on the X-ray source. The markers may, for example, be RFID markers with an associated reader device, they may be geometric shapes recognizable by a camera but also, for example, reflectors for a laser distance measurement or the like.

Other features which are considered as characteristic for the invention are set forth in the appended claims.

Although the invention is illustrated and described herein as embodied in a mobile grating-detector arrangement, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic lateral view of an exemplary embodiment of a foldable grating-detector arrangement according to the invention in an unfolded position;

FIG. 2 is a diagrammatic lateral view of the grating-detector arrangement from FIG. 1, but in a folded position;

FIG. 3 is a perspective view of an exemplary embodiment of an X-ray system according to the invention;

FIG. 4 is a perspective view of the X-ray system positioned in relation to a patient in a patient bed from FIG. 3;

FIG. 5 is a perspective view of the patient bed with a guide of an exemplary embodiment of the X-ray system;

FIG. 6 is a perspective view of the patient bed with a supporting plate of an exemplary embodiment of the X-ray system according to the invention; and

FIG. 7 is a basic schematic representation of the prior art of an X-ray interferometer.

### DETAILED DESCRIPTION OF THE INVENTION

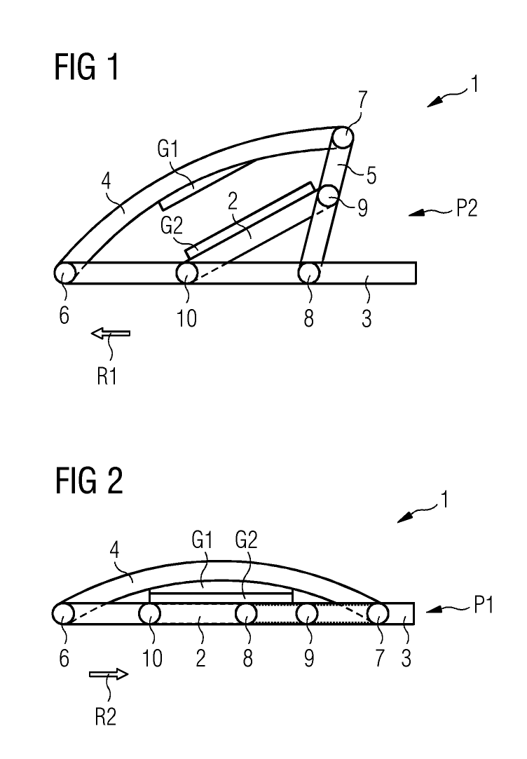

Referring now to the figures of the drawings in detail and first, particularly to FIG. 1 thereof, there is shown a grating-detector arrangement 1 according to the invention in a lateral view by way of example and broadly schematically. The grating-detector arrangement 1 contains a supporting plate 4 and a floor rack 3 which are connected to one another by way of a first swivel joint 6 and pivoted against each other. The first swivel joint 6 is embodied as a hinge here, the supporting plate 4 is flat in design and displays slight curvature away from the floor rack 3. The floor rack 3 contains two lateral struts and a cross member which connects the two lateral struts with one another at the ends such that a U-shape is produced (not shown here). The floor rack 3 is connected to the first swivel joint 6 by way of the two U-arms.

A phase grating G1 is arranged on a side of the supporting plate 4 facing the floor rack 3 in the region of the curvature of the supporting plate 4. On its side opposite the first swivel joint 6, the supporting plate 4 is respectively connected on both sides parallel to the lateral struts of the floor rack 3 by way of a second swivel joint 7 to an end of a supporting strut 5 and pivoted opposite it. At their other end, the supporting struts 5 in each case have a nib 8 facing the lateral struts of the floor rack 3. The nibs 8 of the supporting struts 5 respectively engage in a guide rail embodied on the sides of the lateral struts of the floor rack 3 facing each other. The guide rails form a linear track and at the same time, a pivot bearing for the nibs 8.

In a central area of the supporting strut 5, the supporting strut 5 is connected to a detector 2 by way of a third swivel joint 9 in a corner area of a first side of the detector 2. On a second side facing the first side, on both sides the detector 2 has two nibs 10 facing the lateral struts of the floor rack 3, which like the nibs 8 of the supporting struts 5 engage in the guide rails of the floor rack 3. On a side of the detector 2 facing the supporting plate 4, an analyzer grating G2 is arranged parallel to the detector 2.

The grating-detector arrangement 1 is in an unfolded position P2. In the unfolded position P2, both the phase grating G1, the analyzer grating G2 and the detector 2 are arranged parallel to one another. If the grating-detector arrangement 1 is now moved from the unfolded position P2 to the folded position P1 shown in FIG. 2, the nibs 10 of the detector and the nibs 8 of the supporting strut 5 are moved towards the first swivel joint 6 in a direction R1 inside the guide rails of the floor rack 3. With the movement of the nibs 8, 10, the angles between the detector 2 and the floor rack 3, between the supporting strut 5 and the floor rack 3 and between the supporting plate 4 and the floor rack 3 are also reduced at the same time. This simultaneously results in the third swivel joint 9 and the second swivel joint 7 being moved towards the floor rack 3. In the folded position P1, the swivel joints 6, 7, 8 are arranged on the same level as the floor rack 3. As a result, the supporting plate 4 also lies as flat as possible with its curvature in the region of the floor rack 3. While the gratings G1 and G2 are at a defined interval in the unfolded position P2, in the folded position P1 they lie on top of one other. In the folded position P1, the grating-detector arrangement 1 is therefore flat in comparison with the unfolded position P2. In the folded position P1, the grating-detector arrangement 1 can be advantageously transported and introduced between the patient P and the patient bed B with ease.

To move the grating-detector arrangement 1 from the folded position P1 into the unfolded position P2, the nibs 8, 10 are moved in a direction R2 contrary to the direction R1. This results in precisely the opposite movement pattern, such that the angles between the detector 2 and the floor rack 3 or the supporting strut 5 and the floor rack 3 are increased and the second swivel joint 7 and the third swivel joint 9 are moved away from the floor rack 3. At the same time, the angle between the supporting plate 4 and the floor rack 3 is also increased and the supporting plate 4 deployed. The guide rails of the floor rack 3 may also have detents in which the nibs 8, 10 can engage in order to lock the grating-detector arrangement 1 in the unfolded position P2, but also in intermediate positions between the unfolded position and the folded position P1. In the unfolded position P2, the grating-detector arrangement 1 exhibits the distance defined by the detent between the phase grating G1 and the analyzer grating G2 necessary for interferometric X-ray imaging.

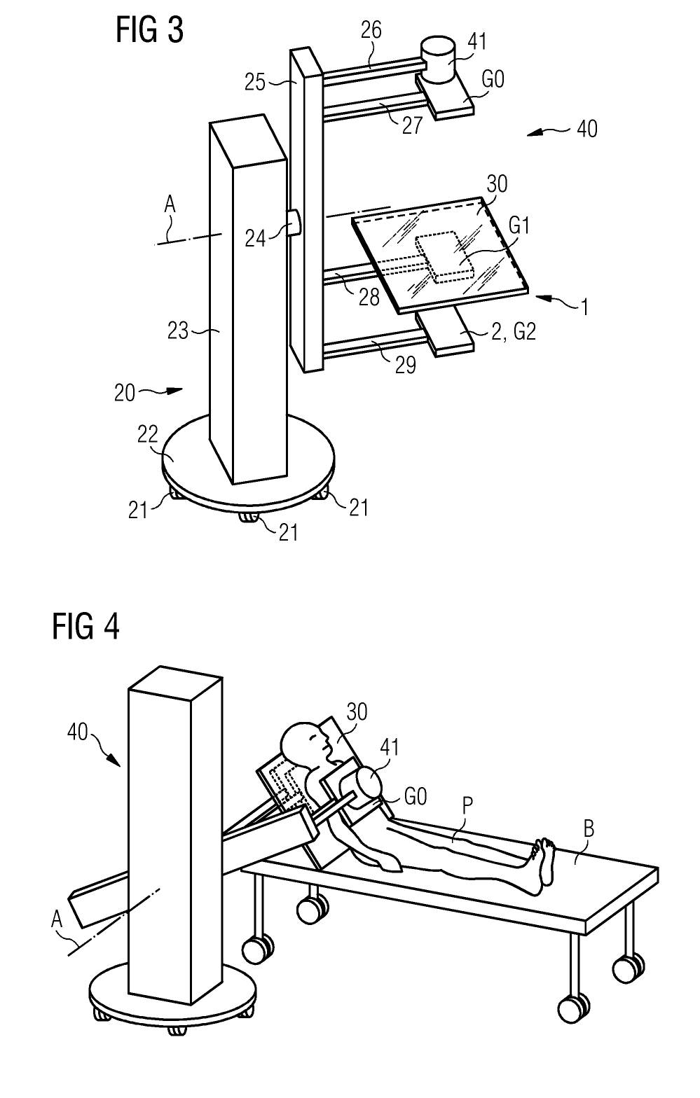

FIG. 3 shows an exemplary embodiment of an X-ray system 40 according to the invention with a further exemplary embodiment of a grating-detector arrangement 1 according to the invention. The grating-detector arrangement 1 contains a trolley 20. The trolley contains a round base plate 22 with two flat sides which is arranged parallel to the ground in operation. The flat sides of the base plate 22 are connected to four casters 21 with the aid of which the X-ray system 40 can be moved. On its other flat side, the base plate 22 is connected to a centrally arranged mounting stand 23 perpendicular thereto. The mounting stand has a pivot bearing 24 by way of which a rotating arm 25 is rotatably connected thereto around a horizontal axis of rotation A.

Further components of the X-ray system 40 are connected to a rotating arm 25 by way of arms 26, 27, 28, 29 which are arranged parallel to the axis of rotation A. An X-ray source 41 is connected to the rotating arm 25 via a first arm 26 and arranged in the region of a first end of the rotating arm 25. It is configured and arranged such that in operation X-rays are essentially emitted from it with a beam path parallel to the rotating arm. An absorption grating GO arranged close to the X-ray source 41 and perpendicular to the beam path follows in the direction of the beam path of the X-ray source 41 and is connected to the rotating arm 25 by way of a second arm 27. The absorption grating GO ensures the coherence of the X-rays in one direction.

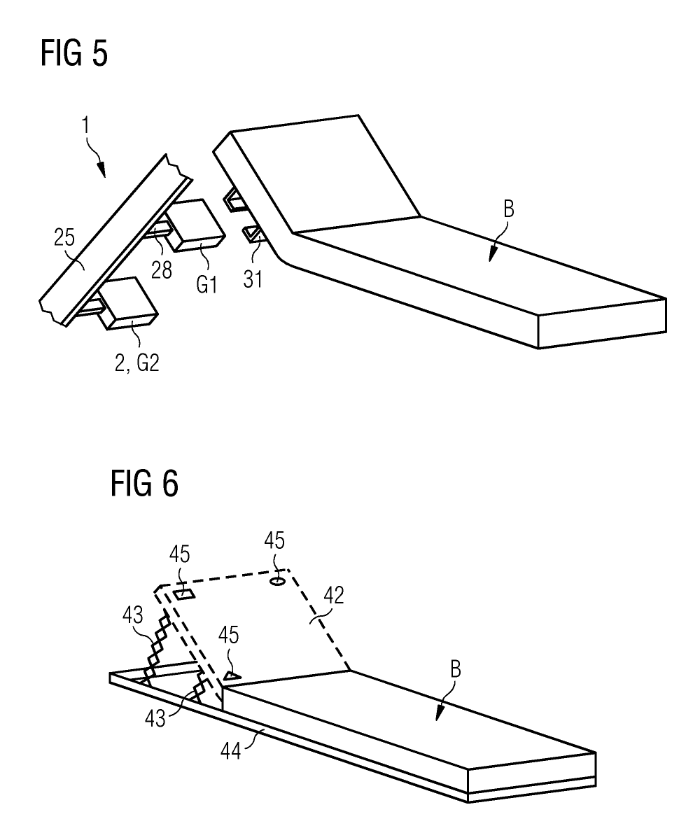

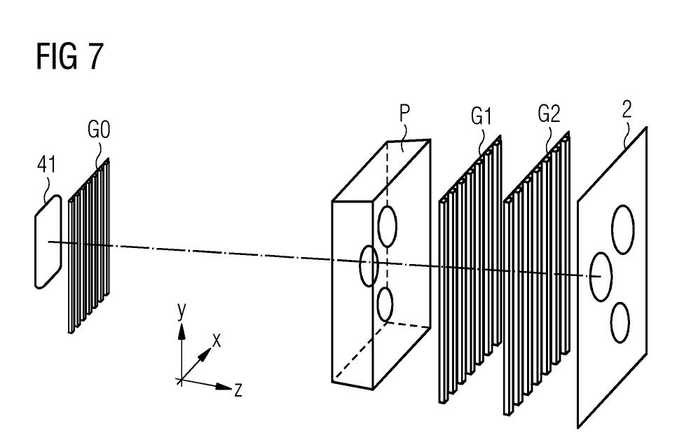

This is followed at a defined interval, in which the X-rays are constructively superimposed, by an optional supporting plate 30 and immediately thereafter by a phase grating G1 which is connected to the rotating arm 25 by way of a third arm 28. The phase grating G1 and the supporting plate 30 are arranged perpendicular to the beam path. The X-ray system 40 can also be used without or without a directly connected supporting plate 30, as explained in more detail with reference to FIG. 5 and FIG. 6. Finally, in the direction of the beam path in the area of the end of the rotating arm 25 opposite the X-ray source 41, there follows an analyzer grating G2 and a detector 2 which are jointly connected to the rotating arm 25 by way of a fourth arm 29.

As aforementioned, interferometric X-ray images of a patient can be created using the X-ray system 40. FIG. 4 shows the X-ray system 40 positioned for an X-ray recording from FIG. 3. A patient P in a patient bed B is supported in a slightly seated position by the supporting plate 30. This results in sufficient space behind the patient for the phase grating G1, analyzer grating G2 and detector 2 required for the recording. The X-ray system according to the invention therefore provides the means to produce an interferometric X-ray image of a patient without the need for larger-scale movement.

FIG. 5 shows a perspective view in section and by way of example of an X-ray system 40 similar to that in FIG. 3 and FIG. 4. The X-ray system 40 here does not include the optional supporting plate 30 but instead a guide 31 is also arranged and attached to the underside on a head part of the patient bed B. The guide 31 is used to record the phase grating G1. It is adjusted to the shape of the phase grating G1 and comprises two corresponding right-angled guide rails. The head part of the patient bed B is used here directly as a means of support for a patient P (not shown here), and the X-ray system 40 is positioned by way of the guide 31. Care must be taken in the area of the head part that a customary foam mattress is not used as a support for the patient P but a mattress comprising material transparent for X-rays.

FIG. 6 shows an exemplary perspective view of a separate supporting plate 42 as part of an X-ray system 40 according to the invention. The supporting plate 42 is flat and two-dimensional in design, such that it can be inserted between the patient P (not shown here) and the mattress with ease. The supporting plate 42 has a lifting mechanism containing four scissor-type lift elements 43. For operation, the scissor-type lift elements 43 are fastened to a frame 44 of the patient bed B using suitable means of attachment. The patient P can then be positioned using the scissor-type lift elements 43 and the head part of the patient bed B removed thereafter. The supporting plate 42 has three markers 45 in the shape of a circle, a square and a triangle to position the remaining components of the X-ray system 40 in relation to the supporting plate 42. The markers can be recorded by a camera (not shown here) arranged on the X-ray source 41. By evaluating the shapes and the positions of the markers in relation to each other, the position of the X-ray source 41 in relation to the supporting plate 42 can then be determined. Instructions can consequently be calculated therefrom, according to which the remaining components of the X-ray system 40 are positioned automatically and/or by the operator.

Finally, it is pointed out once again that the devices and methods previously described in detail are only exemplary embodiments which can be modified in many different ways by a person skilled in the art without departing from the scope of the invention. Furthermore, the use of the indefinite article “a” does not preclude the possibility of the features concerned also being present multiple times. Likewise, the terms “device”, “unit” and “system” do not preclude the component concerned consisting of several interacting subcomponents which may, where applicable, also be spatially distributed.

The following is a summary list of reference numerals and the corresponding structure used in the above description of the invention:
1 Grating-detector arrangement
2 Detector
3 Floor rack
4 Supporting plate
5 Supporting strut
6, 7, 9 Swivel joint
8, 10 Nib
20 Trolley
21 Casters
22 Base plate
23 Mounting stand
24 Pivot bearing
25 Rotating arm
26, 27, 28, 29 Arms
30 Supporting plate
31 Guide
40 X-ray system
41 X-ray source
42 Supporting plate
43 Scissor-type lift elements
44 Frame
45 Marker
A Axis of rotation
B Patient bed
G0 Absorption grating
G1 Phase grating
G2 Analyzer grating
P Patient
P1 Folded position
P2 Unfolded position
R1, R2 Direction

1. A mobile grating-detector configuration, comprising:
an X-ray detector; and
at least one grating configured to record an interferometric X-ray image of at least one body part of a patient in a patient bed in operation.

2. The grating-detector configuration according to claim 1, wherein:
the grating-detector configuration is to be positioned between a flat, folded position and an unfolded position; and
said at least one grating is one of a plurality of gratings and at least some of said gratings are disposed at defined intervals in the unfolded position.

3. The grating-detector configuration according to claim 2, wherein said gratings include a first grating and a second grating, said first grating, said second grating and said X-ray detector are disposed in parallel at least in the unfolded position.

4. The grating-detector configuration according to claim 1, further comprising a trolley connected to said X-ray detector and said at least one grating, with an aid of said trolley, said X-ray detector and said at least one grating can be positioned in relation to the patient.

5. The grating-detector configuration according to claim 4, further comprising a supporting plate for the patient.

6. The grating-detector configuration according to claim 5, wherein said supporting plate has markers to position said supporting plate in relation to an X-ray source.

7. The grating-detector configuration according to claim 5, wherein said supporting plate is connected to said trolley.

8. An X-ray system, comprising:
a mobile grating-detector configuration having an X-ray detector and at least one grating configured to record an interferometric X-ray image of at least one body part of a patient in a patient bed in operation; and
an X-ray source.

9. The X-ray system according to claim 8, wherein:
said grating-detector configuration further has a trolley connected to said X-ray detector and said at least one grating, with an aid of said trolley, said X-ray detector and said at least one grating can be positioned in relation to the patient; and
said grating-detector configuration further has a supporting plate for the patient, said supporting plate has markers to position said supporting plate in relation to said X-ray source, and said supporting plate for the patient is connected to the patient bed in operation.

10. An imaging method, which comprises the steps of:
providing a mobile grating-detector configuration having an X-ray detector and at least one grating configured to record an interferometric X-ray image of at least one body part of a patient in a patient bed in operation; and
using the grating-detector configuration for X-ray interferometric imaging of the patient in the patient bed.

11. The imaging method according to claim 10, which further comprises using the grating-detector configuration for thorax imaging of the patient in the patient bed.
